(19) United States
(12) Patent Application Publication
(10) Pub. No.: US 2013/0048487 A1
(43) Pub. Date: Feb. 28, 2013

(54) SYSTEMS AND METHODS FOR ENRICHMENT AND DETECTION OF PARTICLES

(71) Applicants: Andrea MARZIALI, North Vancouver (CA); Joel PEL, Vancouver (CA); Jason Donald THOMPSON, Vancouver (CA)

(72) Inventors: Andrea MARZIALI, North Vancouver (CA); Joel PEL, Vancouver (CA); Jason Donald THOMPSON, Vancouver (CA)

(73) Assignee: THE UNIVERSITY OF BRITISH COLUMBIA, Vancouver (CA)

(21) Appl. No.: 13/218,124

(22) Filed: Aug. 25, 2011

(51) Int. Cl. G01N 33/559; G01N 27/447; C07C 209/68

(52) U.S. Cl. 204/157.81; 204/462; 204/157.15; 204/456; 204/606

(57) ABSTRACT

Methods and apparatus for separating, concentrating and/or detecting particles are disclosed. The particles are enriched within a separation medium by binding to an immobilized affinity agent. Contaminants and/or undesired particles are washed off the separation medium. The affinity agent is mobilized or the interaction between the particles and the immobilized affinity agent is disrupted. SCODA fields are applied within the medium to separate, concentrate and/or detect a target particle.

### TECHNICAL FIELD

This invention relates to systems and methods for the separation, purification, concentration and/or detection of particles. Embodiments of the invention have application in the purification of particles to remove contaminants and/or undesired particles prior to separation, concentration and/or detection of one or more target particles.

### BACKGROUND

Synchronous coefficient of drag alteration (or “SCODA”) is an approach that may be applied for concentrating and/or separating particles. SCODA may be applied, for example, to DNA, RNA and other molecules. The following background discussion of SCODA is intended to provide examples that illustrate principles of SCODA and is not intended to impose any limitations on the constitution, makeup or applicability of SCODA methods and apparatus generally.

Examples of SCODA are described in the following publications:
U.S. Patent Publication No. 2009/0139867 entitled “Scodaphoresis and methods and apparatus for moving and concentrating particles”;
PCT Publication No. WO 2006/081691 entitled “Apparatus and methods for concentrating and separating particles such as molecules”;
PCT Publication No. WO 2009/094772 entitled “Methods and apparatus for particle introduction and recovery”;
PCT Publication No. WO 2009/001648 entitled “Systems and methods for enhanced SCODA”;
PCT Publication No. WO 2010/051649 entitled “Systems and methods for enhanced SCODA”;
PCT Publication No. WO 2010/121381 entitled “System and methods for detection of particles”;
Marziali, A.; Pel, J.; Bizotto, D.; Whitehead, L. A., “Novel electrophoresis mechanism based on synchronous alternating drag perturbation”, Electrophoresis 2005, 26, 82-89;
Broemeling, D.; Pel, J.; Gunn, D.; Mai, L.; Thompson, J.; Poon, H.; Marziali, A., “An Instrument for Automated Purification of Nucleic Acids from Contaminated Forensic Samples”, JALA 2008, 13, 40-48;
Pel, J.; Broemeling, D.; Mai, L.; Poon, H.; Tropini, G.; Warren, R.; Holt, R.; Marziali, A., “Nonlinear electrophoretic response yields a unique parameter for separation of biomolecules”, PNAS 2008, vol. 106, no. 35, 14796-14801; and
So, A.; Pel, J.; Rajan, S.; Marziali, A., “Efficient genomic DNA extraction from low target concentration bacterial cultures using SCODA DNA extraction technology”, Cold Spring Harb Protoc 2010, 1150-1153,
   each of which is hereby incorporated by reference herein.

SCODA can involve providing a time-varying driving field component that applies forces to particles in some medium in combination with a time-varying mobility-altering field component that affects the mobility of the particles in the medium. The mobility-altering field component is correlated with the driving field component so as to provide a time-averaged net motion of the particles. SCODA may be applied to cause selected particles to move toward a focus area.

Some modes of SCODA exploit the fact that certain particles in appropriate media exhibit non-linear responses to electric fields. In such modes, suitable time-varying electric fields can be used to provide both the driving field and the mobility-altering field to cause certain types of particles to be focused or concentrated at locations within the medium. In some modes, other mobility-altering fields such as temperature are used. In some modes, Joule heating produced by an electric field in a medium is used to alter mobility of particles in the medium.

In many practical cases, the instantaneous velocity of a particle in a medium under the influence of an electric field as the driving field is approximated by:

\vec{v}=μ\vec{E} (1)

where \vec{v} is the velocity of the particle, \vec{E}, is the electric field and μ is the mobility of the particle in the medium given, at least approximately, by:

μ=μ₀+κ|\vec{E}| (2)

where \vec{μ} and κ are constants. Particles with larger values for μ tend to be focused more strongly than particles with smaller values for κ.

SCODA may be applied to concentrate target particles at a location by using a position-dependent mobility-altering field in combination with a driving field which changes in its vector direction. The mobility-altering field may be configured so that for each vector direction of the driving field, target particles upstream from the focus location have higher mobility than target particles downstream from the focus location.

In some cases, SCODA is performed by providing an electrical field having a rotating component and a quadrupole perturbation. The rotating component may be specified, for example, by:

Eₓ=E cos(ωτ) (3)

and

E_{y}=E sin(ωτ) (4)

where E is a magnitude of the electric field component that rotates at an angular frequency ω, and Eₓ and E_{y} are respectively x- and y-components of the rotating electrical field. The perturbing quadrupole component is position-dependent and may be specified, for example, by:

dEₓ=−dE_{q}x cos(2ωτ) (5)

and

dE_{y}=dE_{q}y cos(2ωτ) (6)

where dEₓ and dE_{y} are respectively x- and y-components of the perturbing electrical field, x and y are distances from an origin at the center of the quadrupole field pattern and dE_{q} is the intensity coefficient of the perturbing quadrupole field.

With SCODA fields according to Equations (5) and (6), the average radial velocity of a particle toward the focus location at the origin can be shown to be given by:

$$\overline{\vec{v}} = ( \frac{κ {EdE}_{q}}{4} ) \vec{r} \qquad ( 7 )$$

where \vec{r} is a vector pointing toward the focus location and having a magnitude equal to the distance of the particle from the focus location.

The size of a spot into which particles can be focused depends upon κ as well as on the rate at which the particles can diffuse in the medium as follows:

$$\frac{1}{R} ∝ \sqrt{\frac{κ}{D}} \qquad ( 8 )$$

where R is a radius of the focused spot and D is a diffusion coefficient.

Molecules having large values of \sqrt{κ/D} may focus in the medium under SCODA conditions, and are selectively concentrated within smaller radius distances R relative to molecules with smaller values of \sqrt{κ/D}.

Significant levels of purification can be achieved using SCODA. Some embodiments can achieve purification ratios of 10,000 fold. However, there may be cases where even greater purification is desirable. For example, a large sample volume may contain a small amount of a target particle. There is a need for systems and methods that can achieve greater purification ratios.

The foregoing examples of the related art and limitations related thereto are intended to be illustrative and not exclusive. Other limitations of the related art will become apparent to those of skill in the art upon a reading of the specification and a study of the drawings.

### SUMMARY

One embodiment provides a method for separating one or more target particles from other components of a sample. A medium is provided with one or more affinity agents immobilized within the medium. Each one of the affinity agents has a binding affinity for one or more of the target particles. The sample is loaded on the medium. The one or more target particles are allowed to bind the one or more affinity agents. At least some of the other components are washed out of the medium. The one or more target particles are released and SCODA focusing is performed to concentrate the one or more target particles in the medium.

In some embodiments, the target particles are released by disrupting the binding interaction between the one or more target particles and the affinity agent. The binding interaction may be disrupted by increasing a temperature of the medium. The binding interaction may be disrupted by one or more of increasing a concentration of salt in the medium, increasing a temperature of the medium, increasing the strength of the applied driving field, adding a compound that selectively disrupts the binding interaction to the medium, applying electromagnetic radiation to cause a conformational change in a photochromic moiety that disrupts the binding interaction, or the like. In some embodiments, the affinity agent may be denatured or digested to release the target particles by the addition of a non-specific restriction enzyme or protease.

In some embodiments, the target particles are released by mobilizing the affinity agent. The affinity agent may be immobilized by a labile linkage. The labile linkage may be cleavable by the application of electromagnetic radiation, for example, photo-cleavable. The labile linkage may be acid-cleavable, base-cleavable, fluoride-cleavable, a disulfide bond, or the like. In some embodiments, the affinity agent is mobilized by cleaving a cleavage site within the affinity agent. The cleavage site can be a restriction enzyme cleavage site that can be cleaved by a restriction enzyme, a sequence of amino acids that can be cleaved by a protease, or the like. In some embodiments, the affinity agent is immobilized by a binding interaction with an anchor molecule, and the affinity agent can be mobilized by adding to the medium a compound that disrupts the binding interaction between the affinity agent and the anchor molecule. In some embodiments, a portion of the affinity agent remains bound to the target particles and serves as a handle molecule to enhance SCODA.

One embodiment provides a medium for enhancing the purification ratio obtained by SCODA-based separation. The medium is a medium in which the mobility of target molecules can be varied through the application of a mobility-altering field. A plurality of cleavable affinity agents are immobilized within the medium. One embodiment provides apparatus for conducting SCODA-based separation of particles that includes a medium in which the mobility of target molecules can be varied through the application of a mobility-altering field and a plurality of cleavable affinity agents immobilized within the medium.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A is a schematic diagram of a medium with affinity agents immobilized therein. FIG. 1B shows the medium of FIG. 1A with both target and non-target particles therein. FIG. 1C shows the medium of FIG. 1A after the non-target particles have been washed out of the medium. FIGS. 1D and 1E show the medium after the target particles have been mobilized according to different example embodiments of the present invention.

FIGS. 2A, 2B, 2C, 2D and 2E show schematically exemplary affinity agents that can be used according to different embodiments of the present invention.

FIG. 3A shows a method for performing binding enrichment SCODA-based concentration of one or more target molecules according to one example embodiment of the invention. FIG. 3B shows a method for performing binding enrichment SCODA-based concentration of one or more target molecules according to a different example embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

In some embodiments, SCODA involves providing a time-varying driving field component that applies forces to particles in some medium in combination with a time-varying mobility-altering field component that affects the mobility of the particles in the medium. The mobility-altering field component is correlated with the driving field component so as to provide a time-averaged net motion of the particles that experience a change in mobility in response to the mobility-altering field. In some embodiments, SCODA fields are applied to cause target particles to move toward a focus area. This may be done, for example, by changing a vector direction of the driving field component and adjusting the mobility-altering field component such that mobilities of the target particles upstream from the focus area are higher than mobilities of the target particles downstream from the focus area.

In some embodiments of the present invention, the purification ratio that can be achieved through the application of SCODA fields to particles in a medium is increased by providing one or more affinity agents (i.e. agents which have affinity for one or more target particles) immobilized within the medium, loading a sample, allowing the target particles to bind to the immobilized affinity agents, removing non-target particles from the medium, releasing the target particles, and applying SCODA fields to focus the target particles. In some embodiments, the target particles are released by mobilizing the affinity agent. In some such embodiments, the target particles remain bound to the mobilized affinity agent during the application of the SCODA fields.

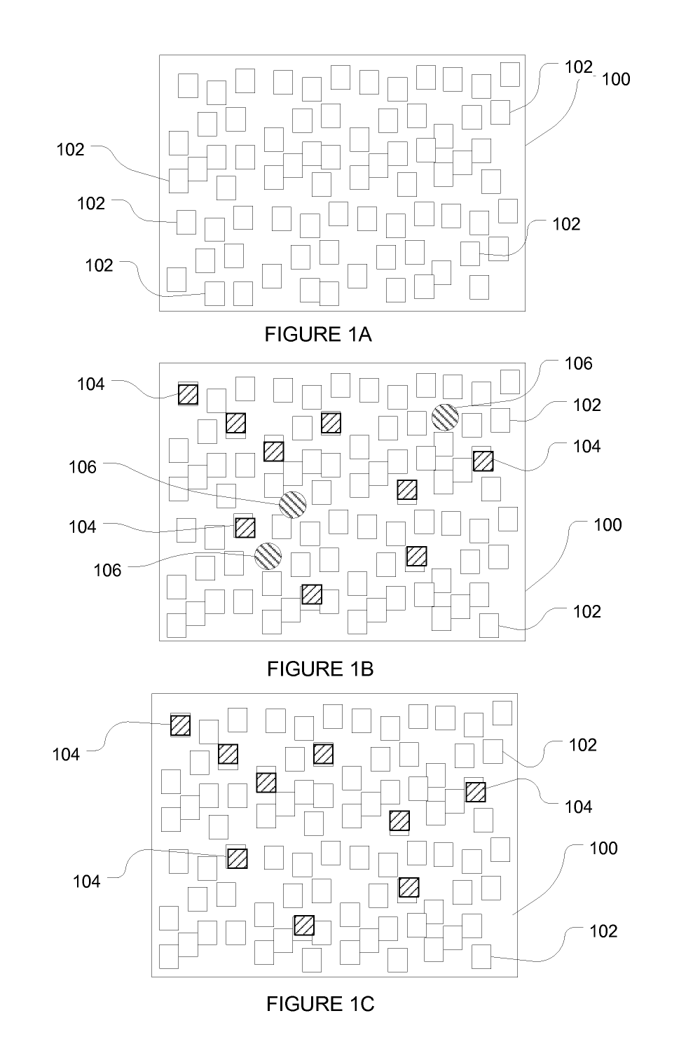

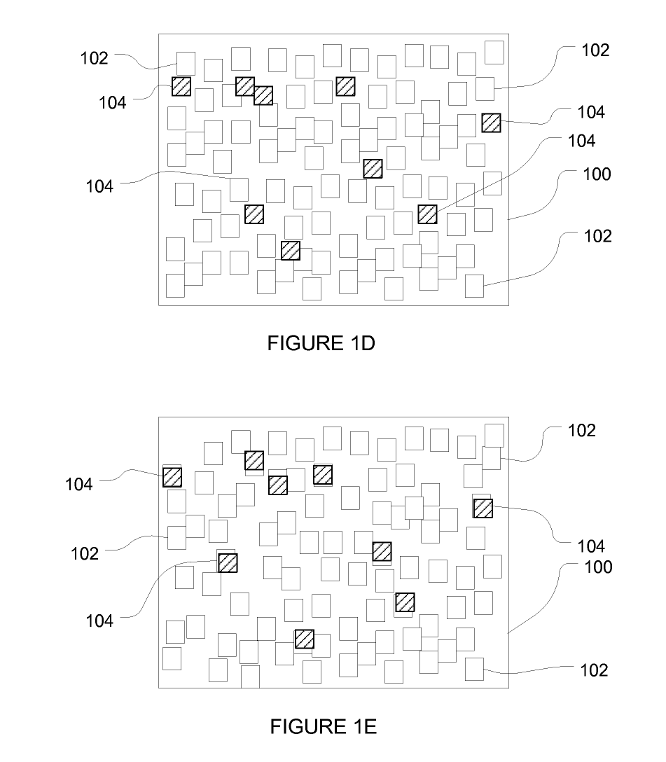

FIG. 1A is a schematic diagram of a medium 100 having immobilized therein a plurality of affinity agents 102. Medium 100 may have any suitable shape and configuration, depending on the particular application.

Any material through which particles can move and in which the mobility of the target particles can be varied in response to a time-varying mobility-altering field may be used to provide medium 100. In some embodiments where the target particles are biomolecules, medium 100 is a polymeric gel, for example an agarose gel, a cross-linked polyacrylamide gel, a linear polyacrylamide gel, or the like. Other potentially suitable media include microfabricated/microfluidic matrices or the like.

Affinity agents 102 are selected to have affinity for the specific target particles of interest. Potentially suitable affinity agents 102 for use in some example embodiments of the present invention include DNA oligonucleotides, RNA oligonucleotides, DNA or RNA aptamers, proteins, antibodies, chemical compounds or other small molecules that bind to the target particle of interest (e.g. molecules such as ethidium bromide or SYBR™ green that can bind DNA), or the like.

The affinity agent 102 may be substantially immobilized within the medium 100 in any suitable manner. For example, where the affinity agent is an oligonucleotide, the oligonucleotide may be covalently bound to the medium, acrydite modified oligonucleotides may be incorporated directly into a polyacrylamide gel, the oligonucleotide may be covalently bound to a bead or other construct that is physically entrained within the medium, or the like.

In embodiments where the affinity agent is a protein or antibody, the protein may be physically entrained within the medium (e.g. the protein may be cast directly into an agarose or polyacrylamide gel), covalently coupled to the medium (e.g. through use of cyanogen bromide to couple the protein to an agarose gel), covalently coupled to a bead that is entrained within the medium, bound to an affinity agent that is directly coupled to the medium or to beads entrained within the medium (e.g. a hexahistidine tag in the protein bound to NTA-agarose), or the like.

Where the affinity agent is a protein that must retain its native conformation to bind the target particle, the conditions under which the medium is prepared and the conditions under which the sample is loaded should be controlled so as not to denature the protein (e.g. the temperature should be maintained below a level that would be likely to denature the protein, the concentration of any denaturing agents in the sample or in the buffer used to prepare the medium, and any field used to introduce target particles from the sample into the medium (e.g. by electrophoresis) should be maintained below a level that would be likely to denature the protein).

Where the affinity agent is a small molecule that interacts with DNA, for example an intercalating agent such as ethidium bromide or SYBR™ green, the compound may be bound to the medium via a linker in any suitable manner. In this context, a linker is a moiety that can attach to both the affinity agent and the medium so as to substantially immobilize the affinity agent with respect to the medium. For example, acryloyl chloride may be used to modify ethidium bromide so that it will bind to an acrylamide gel. Avidin/biotin linkers may be used to couple either SYBR™ green or ethidium bromide to an acrylamide gel.

FIG. 1B is a schematic diagram of medium 100 during or after sample loading. As a sample is loaded onto medium 100, the target particles 104 will bind to the immobilized affinity agents 102. Target particles 104 thereby become largely immobilized in medium 100. Non-target particles or contaminants 106 that do not bind to the immobilized affinity agents 102 (or bind only weakly to affinity agents 102) can be washed from the medium 100 by the application of an appropriate driving force. Conditions under which the driving force is applied are maintained so that target particles 104 remain bound to immobilized affinity agents 102 (and are therefore retained in medium 100), while non-target particles 106 are washed out of medium 100.

FIG. 1C is a schematic diagram of medium 100 after a sample has been loaded and non-target particles 106 have been washed out of the medium. Target particles 104 remain bound to immobilized affinity agents 102. The concentration of target particles 104 relative to non-target particles 106 within medium 100 is thus enriched.

To focus target particles 104 by the application of SCODA fields within the medium 100, the target particles must have some mobility within the medium 100. The target particles 104 cannot be focused while they remain bound to affinity agents 102. Thus, the target particles 104 are mobilized to allow SCODA fields to concentrate the target particles. In some embodiments, the target particles 104 are mobilized by disrupting the binding interaction between immobilized affinity agent 102 and target particles 104. In some embodiments, the target particles 104 are mobilized by mobilizing the affinity agent 102 while the target particles 104 optionally remain bound to the mobilized affinity agent 102.

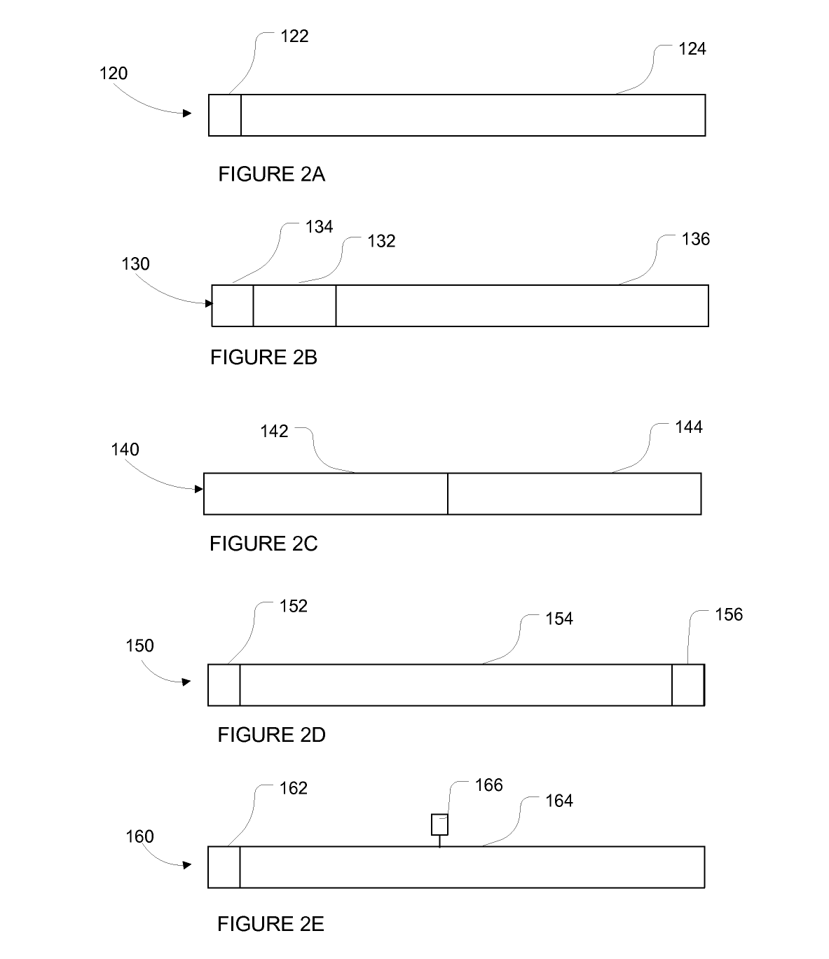

In some embodiments, as shown schematically in FIG. 1D, the target particles 104 are mobilized by disrupting the binding interaction between immobilized affinity agent 102 and target particles 104. The mechanism for disrupting the binding interaction will depend on the nature of the binding interaction. The binding interaction may be disrupted in any suitable manner, for example, by one or more of increasing the temperature of the medium, increasing the applied driving field (which is an electric field in some embodiments), increasing the concentration of one or more salts within the medium, adding an agent that specifically disrupts the binding interaction between the affinity agent and the target particle to the medium (e.g. adding imidazole to the medium in an embodiment where the affinity agent is Ni-NTA agarose and the target particle includes a hexahistidine tag), applying light having an appropriate wavelength to the medium to cause a conformational change in a photochromic group that is positioned to disrupt the binding interaction between the target particle 104 and the affinity agent 102 (e.g. by the application of light having an appropriate wavelength to cause photoisomerization of an azo-benzene group that disrupts the binding interaction in only the trans-configuration or only the cis-configuration) as discussed further with reference to FIG. 2E below, or the like.

In embodiments in which an increase in temperature of the medium is used to disrupt the binding interaction between the affinity agent 102 and the target particles 104, the affinity agent 102 should be selected to provide a binding affinity with the target particle 104 such that the binding interaction can be disrupted within a range of reasonable operating temperatures. For example, the affinity agent 102 can be selected so that the melting temperature of the affinity agent-target particle pairs is within the range of 0° C. to 90° C. The melting temperature of the affinity agent-target particle pairs should be selected so that SCODA fields can be applied to the medium to effect concentration of the target particles at a temperature at which a significant proportion of the target particles 104 are unbound from the affinity agent 102. For example, the melting temperature must be selected to be above a temperature at which the medium freezes, and below a temperature at which the medium boils.

As used herein, the term “melting temperature” means the temperature at which approximately half of the affinity-agent target particle pairs are in the dissociated state and approximately half of the affinity-agent target particle pairs are in the bound state.

In some embodiments, the affinity agent is selected to have little or no binding affinity for one or more non-target particles 106 that are similar in structure to target particles 104. In some embodiments, the affinity agent has some binding affinity for one or more non-target particles, but the melting temperature of the affinity agent-non-target particle duplexes is lower than the melting temperature of the affinity agent-target particle duplexes. In some embodiments, the difference in melting temperature between the target particle-affinity agent duplexes and the non-target particle-affinity agent duplexes is at least 5° C. Non-target particles 106 can be washed from the medium 100 by maintaining the temperature within the medium at a level sufficiently high that the non-target particles 106 do not bind well or at all to the affinity agent 102, but sufficiently low that the target particles 104 spend a significant proportion of time (e.g. greater than 80% of the time) bound to the affinity agents 102.

In some embodiments in which the SCODA fields are applied under conditions where the target particle 104 continues to spend at least a portion of the time (e.g. at least 20% of the time) bound to the immobilized affinity agent 102, greater separation may be achieved. In some such embodiments, the difference in binding energy between the target particle 104 and the affinity agent 102 and one or more similar non-target particles and the affinity agent 102 may be as small as ½ k_{B}T, where k_{B} is Boltzmann's constant and T is the absolute temperature.

In some embodiments in which the target particles 104 are nucleic acids and the affinity agent 102 is also a nucleic acid, affinity agent 102 is selected to have at least five bases that are a perfect complement to a portion of the target nucleic acid sequence. In some embodiments in which the affinity agent 102 is a nucleic acid that also serves as a handle molecule to enhance the selectivity of focusing that can be achieved by the application of SCODA fields, an anchor molecule that remains immobilized during the application of SCODA fields is provided that has at least five bases that are a perfect complement to the sequence of the nucleic acid handle.

In some embodiments, the affinity agent 102 may be denatured and/or digested to release the target particles 104. Denaturation and/or digestion of the affinity agent 102 disrupts the binding interaction between the affinity agent and the target particles 104, thereby releasing the target particles 104. For example, in embodiments where either or both of the affinity agent and the target particle is a protein, the protein can be denatured to mobilize the target particles. In embodiments where the affinity agent is a protein and the target particle is not susceptible to a protease (e.g. the target particle is not a protein), a non-specific protease may be added to the medium to digest the affinity agent. In some embodiments, for example where the affinity agent is a protein, the affinity agent may be denatured through an increase in temperature. In embodiments where the affinity agent is a nucleic acid and the target particle is not susceptible to a nuclease (e.g. the target particle is not a nucleic acid, or is not the same type of nucleic acid as the affinity agent), a non-specific DNase or RNase may be added to the medium to digest the affinity agent.

In some embodiments, as shown schematically in FIG. 1E, the affinity agent 102 is mobilized, thereby mobilizing the target particles 104. In some embodiments, the mobilized affinity agent 102 or a portion thereof remains bound to the target particles 104 during the application of SCODA fields. In some embodiments, the linkage between the affinity agent and the medium is cleavable to mobilize the affinity agent. In some embodiments, the affinity agent includes a cleavable portion to mobilize part of the affinity agent. In some embodiments, a binding interaction between the affinity agent and the medium (which may be a binding interaction between the affinity agent and a second molecule immobilized within the medium) is disrupted to mobilize the affinity agent. SCODA fields can then be applied to focus target particle 104 while target particle 104 optionally remains bound to a mobilized affinity agent 102 or a portion thereof.

FIGS. 2A-2E show affinity agents of different types that can be used in some embodiments of the present invention. In the embodiment shown in FIG. 2A, a first affinity agent 120 is shown schematically. Affinity agent 120 includes a cleavable linkage 122 which initially couples affinity agent 120 to the medium, or to a bead or other substrate entrained within the medium, and a binding region 124 that has a binding affinity for the target particles. Affinity agent 120 may include additional portions or regions at any location (e.g. upstream of, downstream of, and/or interposing cleavable linkage 122 and binding region 124) that do not interfere with the function of cleavable linkage 122 or binding region 124. Linkage 122 may comprise a cleavable covalent linkage for example. Examples of potentially suitable cleavable covalent linkages 122 include linkages that are photolabile (e.g. a 1-(2-nitrophenyl)ethyl moiety, such as the molecules described by Olejnik et al. (Proc. Natl. Acad. Sci. 29(16):7590-4, 1995), hereby incorporated by reference herein), acid or base labile, fluoride cleavable (e.g. a fluoride-cleavable phosphoramidite as described by Fang and Bergstrom (Nucl. Acids Res. 31(2):708-715, 2004), hereby incorporated by reference herein, could be used to immobilize a DNA affinity agent), disulfide linkages, or the like. The cleavable linkage 122 can be cleaved by addition of the appropriate cleaving agent, e.g. by exposing the medium to light having a wavelength suitable to cleave a photolabile linkage (e.g. ultraviolet light), by changing the pH of the medium, for example, by adding acid or base to the medium, by adding fluoride ions, by adding a reducing agent (e.g. dithiothreitol), or the like.

FIG. 2B shows schematically an example affinity agent 130 wherein the affinity agent itself includes a cleavable portion 132. In some embodiments, first portion 134 of affinity agent 130 is configured to link to the medium in any suitable manner. In some embodiments, first portion 134 is covalently linked to a bead or other substrate that is entrained within the medium. In other embodiments, first portion 134 is covalently linked directly to the medium. A second portion 136 of affinity agent 130 has a binding affinity for the target particle. A cleavable portion 132 releasably couples first and second portions 134 and 136. Activation of the cleavable portion will release second portion 136 together with any bound target particle from being anchored in the medium. Affinity agent 130 may include other portions or regions at any location (e.g. upstream of, downstream of, and/or interposing cleavable portion 132, first portion 134, and/or second portion 136) that do not interfere with the function of cleavable portion 132, first portion 134 or second portion 136.

In some embodiments affinity agent 130 comprises an oligonucleotide. In some such embodiments, cleavable portion 132 is a restriction site. The restriction site may be cleaved by addition of a corresponding restriction enzyme to the medium under conditions at which the restriction enzyme is active. The restriction enzyme can cut the oligonucleotide at the restriction site to mobilize the second portion 136 of the affinity agent together with the bound target particle.

In some embodiments, affinity agent 130 is a protein. In some such embodiments, the cleavable portion 132 comprises a specific sequence that is susceptible to cleavage by a sequence-specific protease. Addition of the protease to the medium under conditions in which the protease is active can cleave the cleavable portion 132, thus mobilizing the second portion 136 of the affinity agent and the bound target particle.

FIG. 2C shows schematically an example of an affinity agent 140 that can be immobilized within the medium by a binding interaction with an anchor molecule that is immobilized within the medium. The anchor molecule may be immobilized within the medium in any suitable manner or linked to other anchor molecules in the medium, for example by being entrained within the medium, covalently linked to the medium, covalently linked to a bead or other molecule entrained within the medium, or the like. In such embodiments, the affinity agent includes a portion 142 that binds to the anchor molecule and a portion 144 that binds to the target particle. Affinity agent 140 may be mobilized by the addition of an agent that disrupts the interaction between portion 142 and the anchor molecule. For example, where the affinity agent 140 is a protein, portion 142 may be an affinity tag, for example a hexahistidine tag. The anchor molecule may be a molecule with a binding affinity for a hexahistidine tag, such as NTA-agarose. Affinity agent 140 may be mobilized together with the bound target particle by adding imidazole to the medium in a concentration sufficient to disrupt the interaction between the hexahistidine tag 142 and the anchor molecule. In such an embodiment, the anchor molecule may be NTA-agarose beads or other suitable agent to which the hexahistidine tag binds. Affinity agent 140 may include other portions or regions at any location (e.g. upstream of, downstream of, and/or interposing portion 142 and portion 144) that do not interfere with the function of portions 142 or 144.

In some embodiments in which the target particle is mobilized by mobilizing the affinity agent, the affinity agent is or includes a fluorescent moiety such as a dye, a fluorescent material, or the like. The dye, fluorescent material or the like remains bound to the target particle during the application of SCODA fields by reason of the interaction between the affinity agent and the target particle, and can assist in detection of concentrated target particles. For example, as illustrated in FIG. 2D, an affinity agent 150 includes a cleavable portion 152 for coupling the affinity agent to the medium or a bead or other substrate entrained within the medium, a binding region 154 that has a binding affinity for the target particle of interest, and a fluorescent moiety 156. Fluorescent moiety 156 could be, for example, a rhodamine, fluorescein or cyanine derivative where affinity agent 150 is a nucleic acid or protein, or green fluorescent protein where affinity agent 150 is a protein. The location of fluorescent moiety 156 is not critical, so long as the presence of fluorescent moiety 156 does not interfere with the cleavage of cleavable portion 152 or with the binding interaction between binding region 154 and the target particle of interest. Affinity agent 150 may also include other portions or regions at any location (e.g. upstream of, downstream of, and/or interposing cleavable portion 152, binding region 154 and/or fluorescent moiety 156) that do not interfere with the respective functions of these regions.

FIG. 2E illustrates schematically an affinity agent 160 that includes a photochromic moiety 166 positioned within a region 164 that binds to the target particle. Photochromic moiety 166 is positioned so that it will disrupt the binding interaction between region 164 and the target particle when photochromic moiety 166 undergoes a conformational change caused by the application of electromagnetic radiation of an appropriate wavelength. For example, photochromic moiety 166 may be an azobenzene group or a derivative thereof that disrupts the binding interaction when it changes from the trans-configuration to the cis-configuration. Azobenzene can isomerize from the trans-configuration to the cis-configuration upon exposure to UV light (approximately 300 nm to 400 nm in wavelength), and reverts back to its trans-configuration when exposed to light having a wavelength greater than about 400 nm. Affinity agent 160 is optionally coupled to the medium or to a bead or other substrate entrained within the medium via a region 162, or is otherwise immobilized within the medium. Region 162 need not be a cleavable region. Affinity agent 160 may also include other portions or regions at any location (e.g. upstream of, downstream of, and/or interposing regions 162 and 164) that do not interfere with the respective functions of these regions or with the isomerization of photochromic moiety 166.

Some embodiments do not depend on the diffusion of an agent into the medium 100 to release the target particles 104. For example, mobilization of the target particles 104 by increasing the temperature to disrupt the binding interaction with the affinity agent 102 or to denature affinity agent 102, or application of light or other electromagnetic radiation to cleave a linker to mobilize affinity agent 102 or to cause a change in conformation of a photochromic moiety that disrupts the binding interaction between affinity agent 102 and target particles 104, does not require agents to diffuse into the gel to mobilize the target particles.

It will be apparent to those skilled in the art that the different cleavage mechanisms described above could be combined with one another in various embodiments of the invention. In some embodiments, two different affinity agents (a first affinity agent and a second affinity agent) could be used to initially bind first and second target particles in the medium. The first target particle could be specifically mobilized and SCODA fields could be applied to concentrate the first target particle, while the second target particle remains bound to the second affinity agent. The first target particle could then be extracted or washed out of the medium, and the second target particle could be mobilized and SCODA fields could be applied to concentrate the second particle. For example, the first and second affinity agents could be nucleic acids with different restriction enzyme sites contained therein, the first affinity agent could be a protein while the second affinity agent could be a nucleic acid, or the like. Any combination of affinity agents that would allow the first and second target particles to be selectively immobilized and subsequently released could be used.

After the target particles have been mobilized, SCODA fields (i.e. a time-varying driving field in combination with a time-varying mobility-altering field) can be applied to the medium to focus the target particles. In some embodiments, the mobility-altering field is an electric field. Other potentially suitable mobility-altering fields include time-varying pH, light or other radiation, magnetic fields, an acoustic signal, cyclic chemical changes to the medium, or the like.

In some embodiments, the driving field is an electric field. Other potentially suitable driving fields include a magnetic field, a varying flow of the medium, a varying density gradient of some species in the medium, a gravitational or acceleration field, combinations of any of these or the like.

In some embodiments, both the driving field and the mobility-altering field are electric fields. In some embodiments, both the driving field and the mobility-altering field are provided by a rotating dipole electric field with a quadrupole perturbation. The application of the SCODA fields is controlled so that target particles that are located at a relatively farther distance from a focus spot experience a greater net motion towards the focus spot than away from the focus spot, while target particles that are relatively nearer the focus spot experience a smaller degree of net motion towards the focus spot. For example, the mobility-altering field and/or the driving field may be provided as a spatial gradient within the medium. The application of the time-varying mobility-altering field and the time-varying driving field is correlated so that target particles experience a higher net velocity towards a focus spot when the particles are located a farther distance away from the focus spot within the medium. This causes a net movement of target particles toward a focus spot.

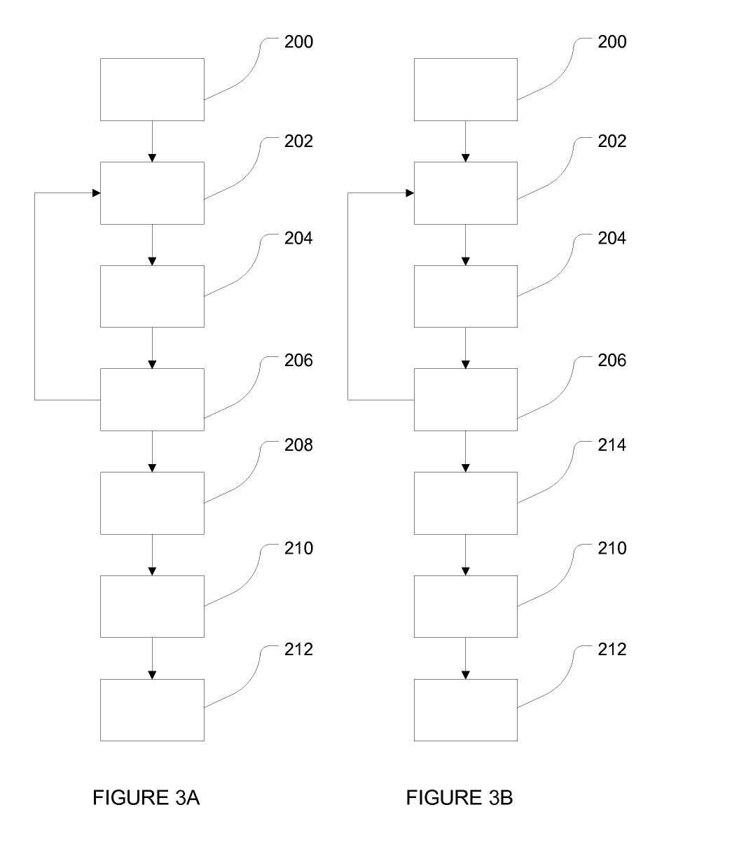

FIG. 3A is a flow chart illustrating a method of enriching target molecules within a medium prior to the application of SCODA fields to focus the target molecules. At box 200, one or more affinity agents are immobilized within a medium. At box 202, a sample is injected into the medium. In some embodiments, the sample may be a portion of a bodily fluid or tissue (for example, blood, urine, a tumor biopsy, or the like) from a mammalian subject, in which the target particle is present at a very low concentration. In some embodiments, the sample may be an environmental sample (e.g. for forensic analysis) in which the target particle is present at a very low concentration. In some embodiments, the sample is optionally treated to lyse cells contained therein and/or to denature the target particle before the sample is injected into the medium.

Injecting particles into the medium may, for example, comprise, placing a liquid sample in a reservoir adjacent to the medium and applying an electrical or other field to urge target particles from the reservoir into the medium.

At box 204, the target molecules encounter and bind to the immobilized affinity agents. At box 206, one or more types of non-target molecules are washed out of the medium by application of an appropriate driving field. Optionally, further sample is injected into the medium at box 202, the target molecules in the further sample are permitted to bind the immobilized affinity agents at box 204, and one or more non-target molecules are washed out of the medium by application of an appropriate driving field at box 206. In some embodiments, the driving field applied to wash the one or more non-target molecules out of the medium is the same field used to inject the sample. In some embodiments, the steps of injecting sample into the medium at box 202, permitting the target molecules to bind the immobilized affinity agents at box 204, and washing the one or more non-target molecules out of the medium by application of an appropriate driving field are performed as a continuous process.

At box 208, the bound target particles are released. This may be done by disrupting the binding interaction between the target molecules and the affinity agent in any suitable manner, for example, by one or more of increasing the strength of the applied driving field, increasing the temperature of the medium, increasing a salt concentration of the medium, adding an agent to the medium to disrupt the binding interaction between the target molecules and the affinity agent, or the like.

In some embodiments in which temperature is used to release the bound target particles, sensors for monitoring the temperature are provided at various locations within or adjacent to the medium. In some embodiments, a controller is provided to receive information from the temperature sensors and control the application of heating, cooling, and/or electric field to different regions of the medium to maintain a desired temperature profile.

In some embodiments the same molecule serves as an affinity agent to hold target particles immobilized and as part of a mobility-altering mechanism. In some embodiments, the binding interaction between the target molecules and the immobilized affinity agent is only partially disrupted. That is, conditions are maintained so that mobility of the target particles is affected by the presence of the affinity agent. For example, the target particle may be bound to the immobilized affinity agent in one or more periods during one cycle of the SCODA fields (i.e. affinity SCODA is performed), thereby further enhancing selectivity for the target particle. In another embodiment a portion of the affinity agent remains bound to the target particles and this portion of the affinity agent has affinity for either another portion of the affinity agent which remains immobilized or to a second affinity agent anchored in the medium. Such particles accordingly serve as a molecular “handle” to enhance the selectivity of SCODA for the target particle.

In some embodiments in which the affinity agent remains immobilized within the medium, the substance used to provide the medium used may be one in which the target particles have a higher mobility than would ordinarily be used to separate the target particles. The affinity agent decreases the effective mobility of the target particles through the medium when SCODA fields are applied under conditions which allow the affinity agent to interact with the target particles for at least a portion of the time (e.g. at least 20% of the time). For example, where the medium is a gel, a gel having a larger pore size can be used in embodiments in which the affinity agent remains immobilized within the medium and SCODA fields are applied under conditions that allow the target particle to interact with the affinity agent than in embodiments in which the affinity agents do not remain immobilized within the medium and/or embodiments in which the SCODA fields are applied under conditions that do not allow the target particle to interact with the immobilized affinity agent. For example, in embodiments in which the medium is an agarose or polyacrylamide gel, a lower percentage of agarose or polyacrylamide may be used to provide a gel that has a larger pore size.

At box 210, SCODA fields are applied to focus the mobilized target molecules. In some embodiments, the time-varying driving field is an electric field and the time-varying mobility-altering field is an electric field. In some embodiments, the time-varying driving field is an electric field and the time-varying mobility-altering field is a temperature field. The temperature field may vary spatially. In some embodiments, both the time-varying driving field and the time-varying mobility-altering field are provided by a rotating dipole field with a quadrupole perturbation.

In some embodiments, variable Joule heating across the medium created by an applied electric field is used to provide a time-varying temperature field. In some embodiments, variable Joule heating across the medium is achieved by an electric field combining a first electric field component which acts to propel the target particles and a second electric field component that adds to the first component in the heated area (i.e. the area of higher mobility) more than it does in other areas of the medium. For example, the second electric field component may be directed in a direction that is opposite to or transverse to the first component outside the heated area. For example, the second component may be a quadrupole field. The orientation of application of the first and second electric field components can be varied to concentrate the target particles at a focus location (for example by rotation, randomly switching orientations, or the like).

In some embodiments, temperature sensors disposed within or adjacent to the medium provide feedback to a controller. The controller can optionally be used to regulate the temperature profile within the medium.

At box 212, the focused target molecules are optionally detected and/or extracted from the medium for further analysis.

FIG. 3B is a flow chart illustrating an alternative method for enriching target particles within a medium prior to application of SCODA fields to focus the target molecules. The embodiment illustrated in FIG. 3B is similar to the embodiment illustrated in FIG. 3A, and the same reference numerals have been used to show the same steps. The embodiment illustrated in FIG. 3B differs from the embodiment illustrated in FIG. 3A in that, after the one or more non-target molecules have been washed from the medium at box 206, the target molecules are mobilized by mobilizing the affinity agent at box 214. The affinity agent can be mobilized in any suitable manner, for example as described above with reference to FIGS. 2A-2D.

In some embodiments, after the target particle has been mobilized, the temperature is optionally lowered. SCODA can be conducted under conditions at which SCODA operates most effectively and with high speed.

Embodiments of the present invention may be applied to facilitate detection, concentration or analysis of target particles where there exists an affinity agent that binds to the target particles and can be immobilized within the medium. Embodiments of the present invention may facilitate detection of target molecules that are present in very low quantities in a sample, and/or that are present in only very low proportions relative to other similar contaminating molecules.

In one exemplary embodiment, selection of desired target molecules occurs through binding of target molecules to immobilized oligonucleotide probes in a gel matrix. The oligonucleotide probes are selected to have a higher affinity for the target molecule than for non-target or contaminant molecules. During injection, contaminant molecules bind more weakly to the immobilized oligonucleotide probes than target molecules, and are removed from the medium by application of a driving field. After the contaminant molecules have been washed out of the medium, the bound target molecules can be released (e.g. through an increase in temperature, an increase in the strength of the driving field applied to the gel, cleavage of a restriction site within the matrix-bound oligonucleotide probe, cleavage of a labile linker within the matrix-bound oligonucleatide probe, activation of a photochromic moiety that disrupts the binding interaction between the bound probe and the target molecule, or the like) and focused.

In one exemplary embodiment for concentrating a target DNA molecule having a particular sequence, a sample containing target DNA and non-target DNA and contaminants is obtained. For example, a blood sample may be obtained from a subject, or an environmental sample may be obtained and placed in aqueous solution. Any cells in the sample are lysed. The DNA is denatured (for example through a heat step) and electrophoretically injected into a medium containing a plurality of oliogonucleotide probes as affinity agents. The sequence of the oligonucleotide probes is selected to be complementary to at least a portion of the target DNA sequence, and to contain at least one mismatch with respect to the sequence of the non-target DNA. The conditions under which the sample is loaded (e.g. temperature, injection field strength and ionic strength) are chosen such that, as molecules from the sample enter the medium, the target DNA binds to and is immobilized by the oligonucleotide probes immobilized in the medium while non-target DNA and contaminants are washed through the gel. For example, during injection, the temperature may be sufficiently high that non-target DNA fragments possess only low affinity for the immobilized oligonucleotide probes and do not bind effectively to the probes. The non-target DNA fragments can be washed through the gel during or after loading. The target DNA fragments are captured by the immobilized oligonucleotide probes because the oligonucleotide probes are designed to have a higher binding affinity for the target DNA (i.e. the oligonucleotide probe-target DNA duplexes have a higher melting temperature) by reason of their complementary sequence.

After loading of the sample, the target DNA is mobilized by increasing the temperature of the medium to disrupt the binding interaction between the oligonucleotide probe and the target DNA. For example, the temperature of the medium may be raised above the melting temperature of the oligonucleotide probe-target DNA duplex, but below a level that would result in boiling of the medium. SCODA fields (for example a rotating dipole electric field with a quadrupole perturbation) are then applied to the medium to concentrate the target DNA at a focus location which is optionally at the center of the medium. In some embodiments, a diffusive extraction well may be fabricated at the focus location to facilitate collection of the target DNA.

In another exemplary embodiment, the target particle is a protein. The affinity agent is a biotinylated protein that has a first portion with a binding affinity for the target protein and a second portion comprising a photocleavable linkage that interposes the biotin moiety and the first portion. A sample containing the target protein and other non-target proteins and contaminants is obtained. For example, a blood sample may be withdrawn from a subject. Any cells in the sample are lysed under conditions that retain the native conformation of the target protein, for example by the addition of a mild detergent. Protease inhibitors are optionally added to the sample. The sample is injected into a polyacrylamide gel having a plurality of the affinity agents (i.e. the biotinylated protein) bound to streptavidin beads that are immobilized in the gel.

After loading of the sample, the affinity agent is mobilized by irradiating the medium with light of a suitable wavelength (e.g. ultraviolet light) to cleave the photocleavable linkage. This step mobilizes the affinity agent-target particle pairs from the medium. SCODA fields are then applied to the medium to concentrate the target particles at a focus location which is optionally at the center of the medium. In some embodiments, a diffusive extraction well may be fabricated at the focus location to facilitate collection of the target protein.

In another specific example embodiment, a sample containing DNA is placed in a first reservoir adjacent to one side of a gel medium. The gel medium includes an affinity agent comprising anchored DNA complementary to a sub-sequence of a target DNA. The DNA of the sample is caused to migrate from the reservoir into the gel medium by applying an electric field across the reservoir and gel medium. The target DNA from the sample encounters and binds to the affinity agents in the gel medium. Other sample DNA that does not bind to the affinity agent is carried through the gel medium to a second reservoir on the far side of the gel medium from the first reservoir. The electric field may be applied by applying a potential difference between electrodes in the first and second reservoirs. In some embodiments, after a period of injection, the sample in the first reservoir is replaced with a buffer solution and the electric field is applied for long enough to drive essentially all non-target DNA from the sample out of the gel medium into the second reservoir. As an alternative, the electric field may be reversed after the injection period to drive non-target DNA from the sample back into the first reservoir. At this point, target DNA is immobilized in the medium. The target DNA may then be released and concentrated by SCODA. In an example embodiment SCODA is performed by applying electrical fields to the medium from each of a plurality of different vector directions. While the electrical fields are being applied, portions of the medium that are upstream from a focus point (upstream relative to the direction that target DNA is moved by the electric fields) are heated sufficiently to make the target DNA mobile. Other portions of the medium are kept below a temperature at which the target DNA remains immobilized or has a reduced mobility. By changing the direction of the electric field and periodically or continuously keeping the temperature of a region upstream from the focus point warm enough to mobilize the target DNA, the target DNA may be concentrated at the focus point. Heating of the medium may be provided by Joule heating, operating a heater in thermal contact with the area of the medium to be heated, optical heating or the like. Ideally the electric field directions are spaced apart at least roughly evenly around a circle. For example, the electric field direction may be rotated continuously, in increments of 90 degrees, 60 degrees, 120 degrees or the like. In some embodiments a plurality of 3 or more, preferably 4 or more electrodes are located in buffer chambers spaced apart around a periphery of the medium and electric fields are applied by applying potential differences between the electrodes.

In some embodiments, after target particles have been selectively enriched and concentrated as described above, the particles may be extracted from the medium, for example using the methods described in Patent Cooperation Treaty publication No. WO 2005/072854, and further analysis carried out. For example, where the target particle is DNA, further analysis such as PCR, sequencing, fluorescent detection, or similar methods as known by persons of skill in the art may be conducted on the concentrated target particles.

Some embodiments of the invention provide a medium containing a plurality of cleavable affinity agents immobilized within the medium.

Some embodiments of the invention provide kits for detecting a specific particle of interest. The kit includes a medium within which the mobility of the specific particle of interest can be varied. The medium contains a plurality of cleavable affinity agents having a binding affinity for the particle of interest. The kit includes a reagent that can be added to the medium to mobilize the particle of interest. A user can isolate a sample, optionally lyse any cells and/or denature the specific particle of interest in the sample, load the sample on the medium, wash contaminating particles out of the medium, mobilize the specific particle of interest by adding the reagent to the medium under conditions at which the reagent is active to cleave the linkage immobilizing the affinity agent and/or break the affinity agent apart, and apply SCODA fields to the medium to focus the particle of interest. The particle of interest can then optionally be detected, extracted from the medium and/or subjected to further analysis.

Some embodiments of the invention provide an apparatus for conducting SCODA-based separation of particles. The apparatus comprises a medium containing a plurality of cleavable affinity agents and apparatus for applying and controlling a time-varying driving field and a time-varying mobility field to concentrate target particles in the medium.

While a number of exemplary aspects and embodiments have been discussed above, those of skill in the art will recognize certain modifications, permutations, additions and sub-combinations thereof. It is therefore intended that the scope of the following appended claims and claims hereafter introduced are not limited by the preferred embodiments set forth in the examples, but should be given the broadest interpretation consistent with the description as a whole.

What is claimed is:

1. A method for separating target particles from other components of a sample, the method comprising:
providing a medium comprising an affinity agent immobilized at a plurality of locations within the medium, the affinity agent having a binding affinity for the target particles;
loading the sample on the medium;
allowing the target particles to bind to the affinity agent;
washing at least some of the other components out of the medium;
releasing the target particles; and
applying SCODA fields to focus the target particles in the medium.

2. A method as defined in claim 1, wherein releasing the target particles comprises disrupting a binding interaction between the target particles and the affinity agent.

3. A method as defined in claim 2, wherein disrupting the binding interaction comprises increasing a temperature of the medium.

4. A method as defined in claim 2, wherein the affinity agent comprises a photochromic group positioned to disrupt the binding interaction between the affinity agent and the target particles when the photochromic group undergoes isomerization, and wherein disrupting the binding interaction comprises applying to the gel electromagnetic radiation having a wavelength suitable to cause isomerization of the photochromic group.

5. A method as defined in claim 4, wherein the photochromic group comprises azobenzene or a derivative thereof, and wherein disrupting the binding interaction comprises applying ultraviolet light to the gel.

6. A method as defined in claim 2, wherein disrupting the binding interaction comprises one or more of: increasing a concentration of salt in the medium, increasing a temperature of the medium, and increasing the strength of an applied driving field.

7. A method as defined in claim 2, wherein disrupting the binding interaction comprises adding a compound that selectively disrupts the binding interaction to the medium.

8. A method as defined in claim 2, wherein one or both of the affinity agent and the target particles are a protein and disrupting the binding interaction comprises denaturing the protein.

9. A method as defined in claim 2, wherein disrupting the binding interaction comprises digesting the affinity agent.

10. A method as defined in claim 9, wherein the affinity agent comprises a protein, and wherein disrupting the binding interaction comprises adding a protease to the medium.

11. A method as defined in claim 9, wherein the affinity agent comprises DNA, and wherein disrupting the binding interaction comprises adding a DNase to the medium.

12. A method as defined in claim 9, wherein the affinity agent comprises RNA, and wherein disrupting the binding interaction comprises adding an RNase to the medium.

13. A method as defined in claim 1, wherein the affinity agent is covalently bound to the medium.

14. A method as defined in claim 1, wherein releasing the target particles comprises mobilizing the affinity agent.

15. A method as defined in claim 14, wherein mobilizing the affinity agent comprises cleaving a labile linkage that immobilizes the affinity agent.

16. A method as defined in claim 15, wherein the labile linkage comprises a photo-cleavable linkage, and mobilizing the affinity agent comprises exposing the medium to light having a wavelength suitable for cleaving the photo-cleavable linkage.

17. A method as defined in claim 15, wherein the labile linkage comprises a base-labile linkage, and mobilizing the affinity agents comprises adding a base to the medium.

18. A method as defined in claim 15, wherein the labile linkage comprises an acid-labile linkage, and mobilizing the affinity agent comprises adding an acid to the medium.

19. A method as defined in claim 15, wherein the labile linkage comprises a disulfide bond, and mobilizing the affinity agents comprises adding a reducing agent to the medium.

20. A method as defined in claim 15, wherein the labile linkage comprises a fluoride cleavable bond, and mobilizing the affinity agent comprises adding fluoride ion to the medium.

21. A method as defined in claim 15, wherein the affinity agent contains a cleavage site, and mobilizing the affinity agent comprises activating the cleavage site.

22. A method as defined in claim 21, wherein the affinity agent comprises a nucleic acid, the cleavage site comprises a restriction enzyme cleavage site that can be cleaved by a restriction enzyme, and mobilizing the affinity agent comprises adding the restriction enzyme to the medium under conditions such that the restriction enzyme is active.

23. A method as defined in claim 21, wherein the affinity agent comprises a protein, the cleavage site comprises a sequence of amino acids that can be specifically cleaved by a protease, and the step of mobilizing the affinity agent comprises adding the protease to the medium under conditions such that the protease is active.

24. A method as defined in claim 14, wherein the affinity agent is immobilized by a specific binding interaction between the affinity agent and anchor molecules that are immobilized within the medium, and mobilizing the affinity agent comprises adding a compound that disrupts the binding interaction between the affinity agent and the anchor molecules.

25. A method as defined in claim 1, wherein providing the medium comprises providing a gel.

26. A method as defined in claim 1, wherein the target particles comprise DNA, RNA or protein.

27. A method as defined in claim 1, wherein the applied SCODA fields comprise a rotating dipole electric field with a quadrupole perturbation.

28. A method as defined in claim 14, wherein at least a portion of the affinity agent comprises a fluorescent moiety.

29. A method as defined in claim 1, wherein the steps of loading the sample on the medium, allowing the target particles to bind to the affinity agents, and washing at least some of the other components out of the medium are performed as a continuous process.

30. A medium for enhancing the purification ratio obtained by SCODA-based separation comprising a medium within which the mobility of target particles can be varied, wherein a plurality of cleavable affinity agents that bind to the target particles are immobilized within the medium.

31. Apparatus for conducting SCODA-based separation of target particles comprising a medium as described in claim 30.
